(19) United States
(12) Patent Application Publication
(10) Pub. No.: US 2013/0132105 A1
(43) Pub. Date: May 23, 2013

(54) SYSTEM AND METHOD FOR ASSIGNING WORK STUDIES WITHIN A WORK LIST

(71) Applicants: Cleon Hill Wood-Salomon, St. Jacobs (CA); Marc Durand, Baden (CA); Geoff Wheeler, Breslau (CA); John Delong, Millbank (CA)

(72) Inventors: Cleon Hill Wood-Salomon, St. Jacobs (CA); Marc Durand, Baden (CA); Geoff Wheeler, Breslau (CA); John Delong, Millbank (CA)

(21) Appl. No.: 13/298,688

(22) Filed: Nov. 17, 2011

(51) Int. Cl. G06Q 50/22

(52) U.S. Cl. 705/2

(57) ABSTRACT

A system and method for assigning work studies to be read within a work list are disclosed. In some embodiments, the systems and methods may relate to assigning a radiology work study such as a radiology image study to be read within a radiologist work list associated with at least one radiologist. The assigning of the radiology work study to be read within the radiologist work list may comprise assigning an order to the radiology work studies that are within the radiologist work list. The assigning of the radiology work studies may be based on a service level priority for the radiology work studies.

### FIELD

The present disclosure relates to a system and method for assigning work studies. In some embodiments, the present disclosure relates to a system and method for assigning work studies within a work list.

### BACKGROUND

Conventional radiology departments typically allocate work based on proximity of professionals to a hospital. For example, a radiology department of a specific hospital may have a certain number of radiologists. Radiology work that originates from patients at the specific hospital may be conventionally assigned to the various radiologists or medical professionals employed at the specific hospital's radiology department.

However, recent trends in the medical industry have involved the merging of various medical practice groups or departments. For example, radiology practice groups of a first hospital have been merged with radiology practice groups of one or more other hospitals or medical groups. As such, radiology practice groups may now comprise a plurality of medical professionals (e.g., radiologists) over a plurality of locations (e.g., hospitals).

As such, it is desirable to develop systems and methods of generating a workflow. The workflow that is generated may be based on medical professional parameterization as well as other parameters in order to address the above-described trends in the medical industry.

### SUMMARY

The present disclosure introduces systems and methods for assigning work studies to be read within a work list.

The system or method may comprise a work flow comprising the assigning of radiology work studies to be read within a radiologist work list. For example, a radiologist work list may be received. In some embodiments, the radiologist work list may comprise one or more radiology work studies (e.g., radiology images to be read by a radiologist). One or more parameters of the radiology work studies in the radiologist work list may be received. In some embodiments, the parameters may comprise a service level priority for the radiology work studies. An order for the radiology work studies within the radiologist work list may be assigned. In some embodiments, the assigning of the order of the radiology work studies may be based on the received parameter associated with the radiology work studies.

In some embodiments, the order of the radiology work studies may be assigned such that the order specifies a priority for the radiology work studies to be read by a radiologist associated with the radiologist work list. The ordering may further be based on a parameter of the radiologist. In some embodiments, the parameter of the radiologist may comprise at least one of a subspecialty of the radiologist, a professional credential of the radiologist, and a location or jurisdiction of the radiologist.

### BRIEF DESCRIPTION OF THE DRAWINGS

The novel features of the disclosure are set forth in the appended claims. However, for purpose of explanation, several embodiments of the disclosure are set forth in the following figures.

FIG. 1 illustrates an example environment for generating a workflow based on medical professional parameterization.

FIG. 2 is a flow diagram of an example method for creating a workflow based on parameters of a reading radiologist in accordance with some embodiments.

FIG. 3 is a block diagram of a parameterization filter being used to generate a workflow of radiology work studies in accordance with some embodiments of the disclosure.

FIG. 4 is a block diagram of a work flow for assigning radiology work studies to be read by one or more radiologists.

FIG. 5 is a flow diagram of an exemplary method for assigning radiology work studies to a radiologist work list and to assign the radiologist work lists to be read by a radiologist.

FIG. 6A is a diagram illustrating a graphical user interface for setting radiologist parameters for an assignment filter in accordance with some embodiments of the disclosure.

FIG. 6B is a diagram illustrating a graphical user interface for specifying assignment rules for an assignment filter in accordance with some embodiments.

FIG. 7 is a flow diagram of a method for assigning a radiology work study to a radiologist work list in accordance with some embodiments.

FIG. 8 illustrates an example method for a radiology workflow to assign a radiology work study to either a first radiologist work list or a second radiologist work list.

FIG. 9 illustrates a flow diagram of a method for assigning or ordering radiology work studies to be read within a radiologist work list.

FIG. 10 is an example workflow of assigning an order of radiology work studies within a work list in accordance with some embodiments of the disclosure.

FIG. 11 illustrates an example method for auto escalating a service level priority associated with a radiology work study.

FIG. 12 is an example of a radiology work study with an escalated service level priority and being reordered or reassigned within a radiologist work list.

FIG. 13 illustrates an example workflow of reassigning a radiology work study from a first radiologist work list to a second radiologist work list.

FIG. 14 illustrates an example method for reassigning a radiology work study to another radiologist work list in accordance with some embodiments of the disclosure.

FIG. 15 illustrates an example method for determining whether to keep a radiology work study in a radiologist work list or to reassign the radiology work study from the radiologist work list to a second radiologist work list.

FIG. 16 illustrates a graphical user interface comprising a plurality of work lists in accordance with some embodiments of the disclosure.

FIG. 17 depicts a diagram illustrating an exemplary computing system environment for execution of the operations comprising various embodiments of the disclosure.

### DETAILED DESCRIPTION

The systems and methods disclosed herein relate to assigning work studies to be read within a work list.

In the following detailed description, numerous specific details are set forth in order to provide a thorough understanding of the present disclosure. However, it will become obvious to those skilled in the art that the present disclosure may be practiced without these specific details. The description and representation herein are the common means used by those experienced or skilled in the art to most effectively convey the substance of their work to others skilled in the art. In other instances, well known methods, procedures, and systems have not been described in detail to avoid unnecessarily obscuring aspects of the present disclosure.

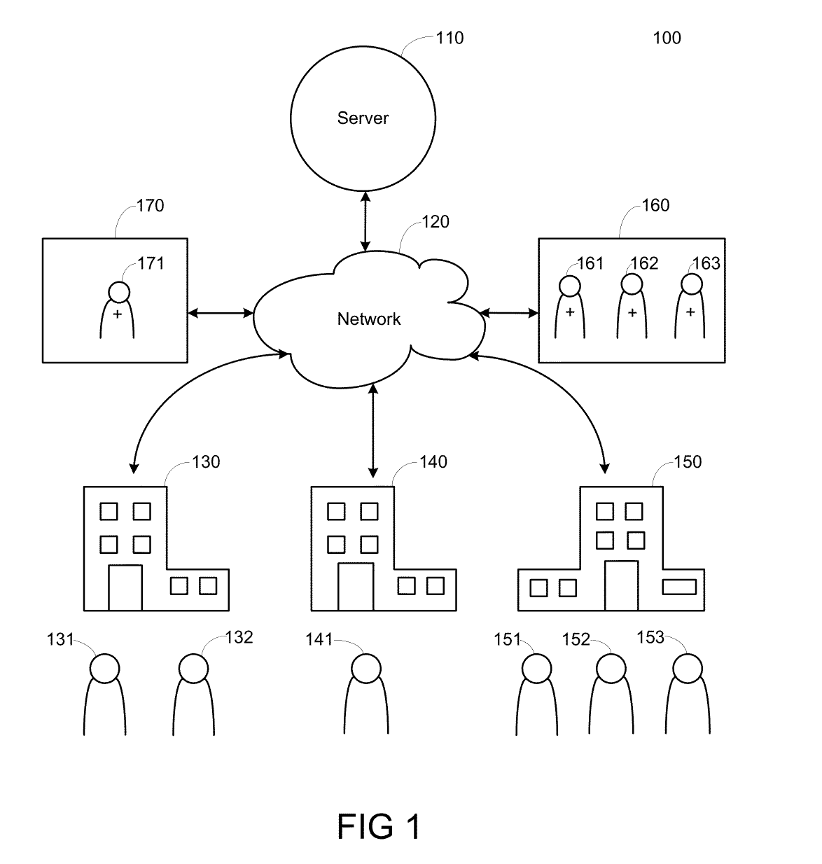

FIG. 1 illustrates an example environment 100 for generating a workflow based on medical professional parameters. In general, the environment 100 may comprise a plurality of medical locations and a plurality of medical professionals such that work from the medical locations may be filtered or assigned with respect to medical professional parameters to generate a workflow of the work for the medical professionals.

As seen in FIG. 1, the environment 100 may comprise a server 110, medical professional locations 160 and 170, and patient locations 130, 140, and 150. Each of the server 110, medical professional locations 160 and 170, and the medical patient locations 130, 140, and 150 may be connected to or coupled to a network 120 (e.g., the Internet) such that each medical professional location and/or medical patient location may communicate with server 110 over the network 120 or between any two locations (e.g., a medical patient location communicating with a medical professional location, a medical professional location communicating with another medical professional location, etc.).

In some embodiments, each of the medical patient locations 130, 140, and 150 may be associated with one or more patients (e.g., a patient associated with a radiology image study or a radiology work study). For example, each of the medical patient locations 130, 140, and 150 may be a hospital, doctor's office, or any other medical practice location. In some embodiments, medical patient locations 130, 140, and 150 may each correspond to a different location and/or jurisdiction. For example, medical patient location 130 may be in a first state (e.g., the state of Washington), medical patient location 140 may be in a second state (e.g., the state of Oregon), and medical patient location 150 may be in a third state (e.g., the state of California). As such, each of the medical patient locations 130, 140, and 150 may be in a different jurisdiction and/or different location. Each of the medical patient locations 130, 140, and 150 may be associated with one or more medical patients. For example, medical patient location 130 may be associated with medical patients 131 and 132, medical patient location 140 may be associated with medical patient 141, and medical patient location 150 may be associated with medical patients 151, 152, and 153. In some embodiments, each of the medical patients 131, 132, 141, 151, 152, and 153 may be associated with at least one medical work study. For example, each of the medical patients may be associated with a radiology work study (e.g., a radiology image), as will be described in further detail below.

As such, each medical location may be associated with one or more medical patients where each patient may be associated with one or more work studies (e.g., radiology studies). The medical locations may be associated with one or more jurisdictions. As such, each work study from each patient may further be associated with the one or more jurisdictions.

As seen in FIG. 1, the environment 100 may further comprise one or more medical professional locations. For example, medical professional location 160 may be in a first state, location, and/or jurisdiction and medical professional location 170 may be in a second state, location, and/or jurisdiction. Each medical professional location may be associated with one or more medical professionals. For example, a medical professional location 170 may be associated with a medical professional 171. Moreover, the medical professional location 160 may be associated with a medical professional 161, a medical professional 162, and a medical professional 163. In some embodiments, each of the medical professionals 161, 162, 163, and 170 may be a radiologist. As such, in some embodiments, each of the medical professionals 161, 162, 163, and 170 may be associated with one or more parameters used to generate a workflow for radiologists reading radiology work studies, as will be described in further detail below.

As such, in some embodiments, the environment 100 may comprise one or more radiology professional locations that may be associated with one or more radiology patients. In some embodiments, each of the radiology patients may be associated with a radiology work study (e.g., a series of radiology images) that may have originated from or be associated with a particular medical patient location (e.g., a hospital in a particular state). In some embodiments, a radiology work study from a hospital (e.g., medical patient location 130, 140, and/or 150) may be communicated to the server 110 over the network 120. For example, a radiology patient (e.g., medical patient 131, 132, 141, 151, 152, and/or 153) may be associated with or have a radiology work study done at a hospital. Each of the radiology work studies from the radiology patients may be communicated or sent to the server 110 over the network 120. In some embodiments, the server 110 may store each of the radiology work studies into a database and/or a repository of radiology work studies. Moreover, in some embodiments, the server 110 may assign, relay, communicate, and/or send one or more of the radiology work studies received from one or more hospitals to at least one radiologist professional location (e.g., medical professional location 160 and/or 170) or to a specific radiologist (e.g., medical professional 161, 162, 163, and/or 171). In some embodiments, the assigning of a radiology work study to a radiologist may be based on at least one parameter of the radiologist. Moreover, the server 110 may further assign an order to radiology work studies that have been assigned to a radiologist, as will be discussed in further detail below.

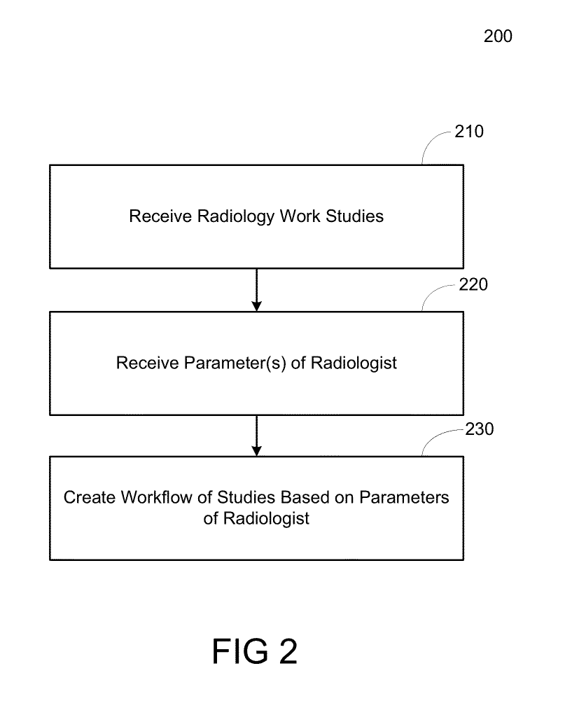

FIG. 2 is a flow diagram of an example method 200 for creating a workflow based on parameters of a radiologist (e.g., a reading radiologist). In general, the method may receive one or more work studies and assign the one or more work studies to a work list associated with a radiologist.

As seen in FIG. 2, at block 210, a work study may be received. For example, a one or more radiology work studies may be received. Each of the radiology work studies may be received from a hospital (e.g., medical patient location 130, 140, and/or 150) and be associated with a radiology patient (e.g., medical patients 131, 132, 141, 151, 152, and/or 153). In some embodiments, the server 110 may receive the radiology work studies. In some embodiments, a radiology work study may be received a plurality of times. For example, the process (e.g., receiving a radiology work study) may be repeated a plurality of times based on updates and/or changes in a radiology work study status, patient conditions, timed event, and/or radiologist updates. As such, as appreciated by one skilled in the art, the receiving of a radiology work study as used within the present disclosure may be considered to be dynamic and a radiology work study may be continuously received.

At block 220, one or more parameters associated with a radiologist (e.g., medical professional 161, 162, 163, and/or 170) may be received. In some embodiments, the one or more parameters may be received from a radiology practice location (e.g., medical professional location 160 and/or 170) and/or may be stored within a memory of the server 110. At block 230, a work list of the received radiology work studies may be created based on at least one parameter of one or more radiologists. In some embodiments, the server 110 may assign or create a work list for one or more radiologists. For example, the method 200 may receive a plurality of radiology work studies and assign a first subset of the plurality of radiology work studies to a first radiologist based on parameters associated with the first radiologist and may further assign a second subset of the plurality of radiology work studies to a second radiologist based on parameters associated with the second radiologist. In some embodiments, the work lists may also be based on parameters associated with the radiology work studies.

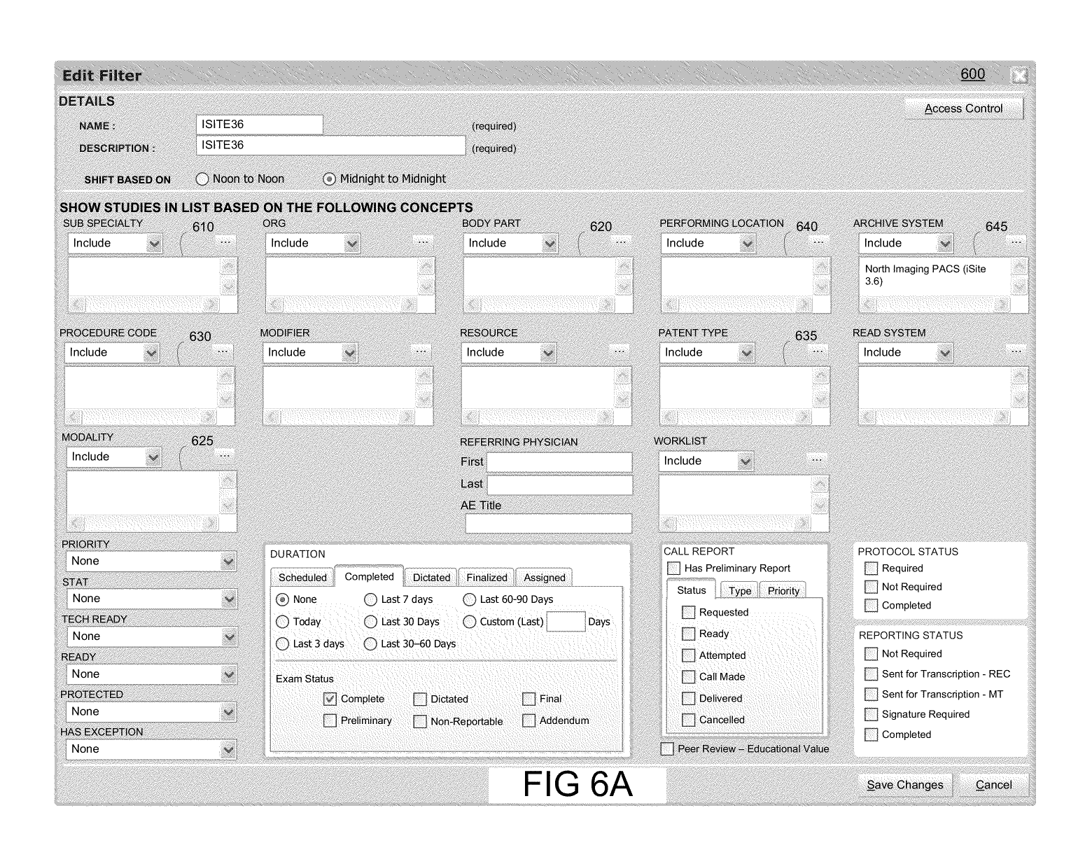

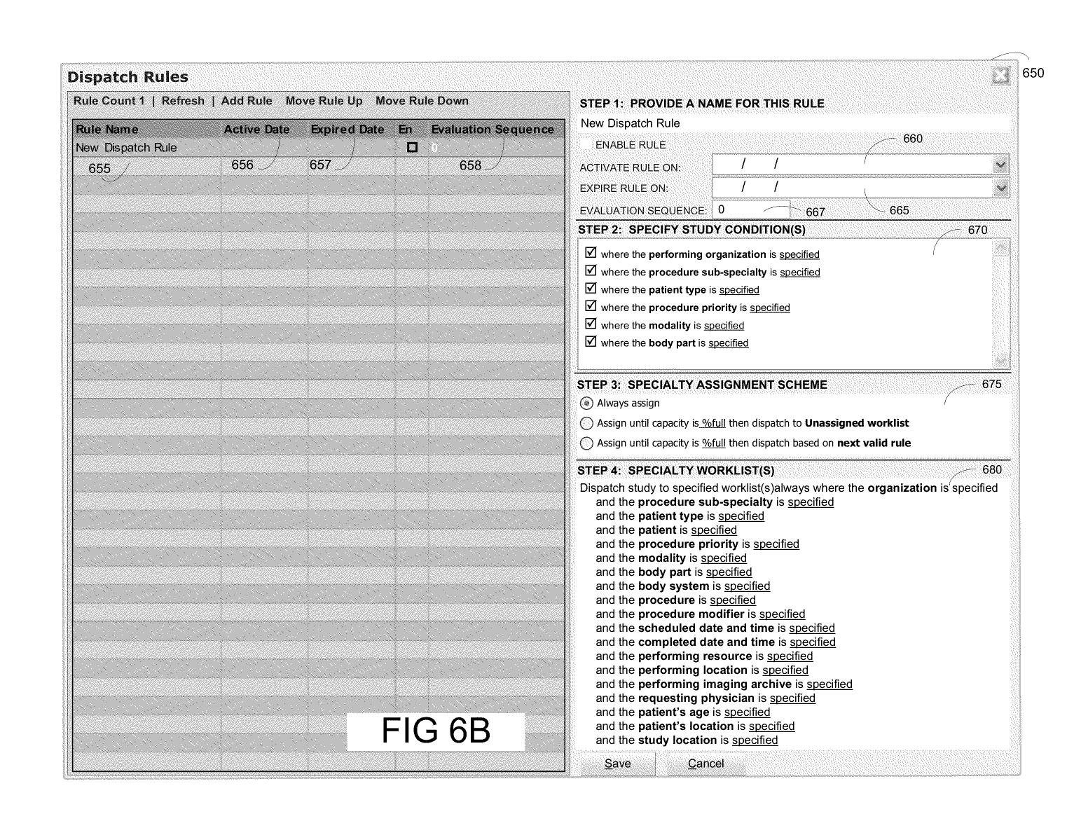

As such, radiology work studies may be assigned to or distributed to one or more work lists associated with one or more radiologists based on characteristics, parameters, and/or credentials of each of the radiologists. Thus, the method 200 may be used to generate a workflow of the radiology work studies. Further details on characteristics, parameters, and/or professional credentials of radiologists are discussed in further detail below with regard to FIGS. 6A and 6B.

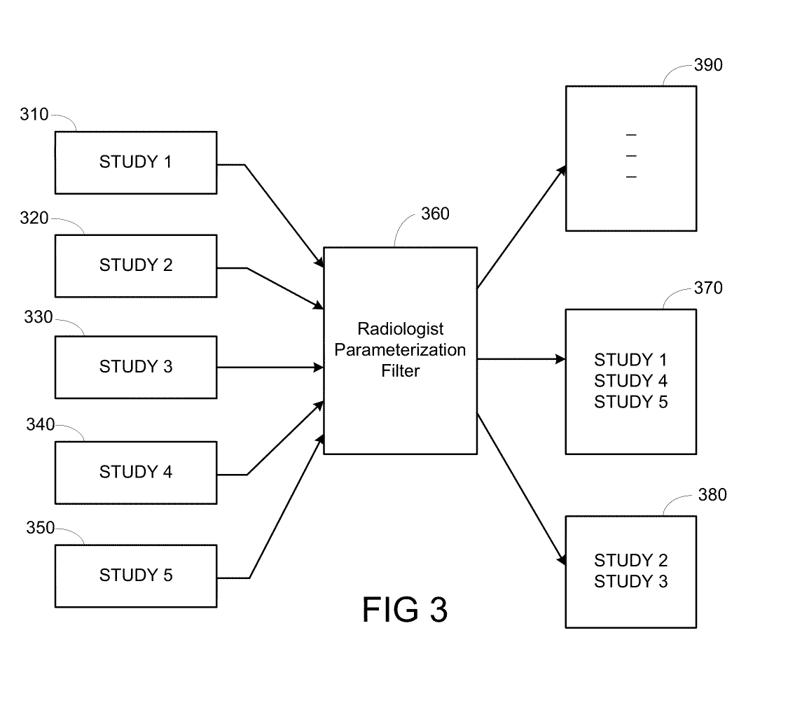

FIG. 3 is a block diagram of a parameterization filter being used to generate a workflow for work studies in accordance with some embodiments of the disclosure. In general, work studies (e.g., radiology work studies) may be received and assigned to a work list associated with a medical professional (e.g., a radiologist) based on parameters of the medical professional. In some embodiments, workflow may comprise a method for assigning work studies (e.g., radiology work studies) to a work list (e.g., a radiologist work list) associated with a medical professional (e.g., a radiologist). In the same or alternative embodiments, a work list (e.g., a radiologist work list) may comprise work studies that the medical professional associated with the work list (e.g., a radiologist) has been assigned and will need to read or complete. Since the workflow comprises assigning work studies to work lists, such a workflow method may be described as a ‘push’ method (e.g., radiologists are actively assigned radiology work studies) as opposed to a ‘pull’ method (e.g., radiologists select radiology work studies on their own to read or complete).

As seen in FIG. 3, a plurality of work studies 310, 320, 330, 340, and 350 (e.g., work studies received from medical patient location 130, 140, and/or 150) may be received or pooled together. In some embodiments, the work studies may be radiology work studies that comprise a radiology image to be read by a radiologist. Each of the radiology work studies may be run through or analyzed with respect to a radiologist parameterization filter 360. In some embodiments, the radiologist parameterization filter 360 may comprise characteristics, parameters, and/or credentials of one or more radiologists. In the same or alternative embodiments, the radiologist parameterization filter 360 may also comprise parameters of radiology work studies and/or parameters of a patient associated with a radiology work study (e.g., a patient's age, gender, height, weight, clinical history, etc.). Each radiology work studies 310, 320, 330, 340, and 350 may be filtered or analyzed by the radiologist parameterization filter 360 such that each of the radiology work studies 310, 320, 330, 340, and 350 may be assigned to, directed to, or forwarded to a radiologist work list to (e.g., from the server 110 to a medical professional location 160 and/or 170 or to a work list associated with medical professionals 161, 162, 163, and/or 170). As such, the radiologist parameterization filter 360 may use parameters of the radiologist, parameters associated with a radiology work study, and parameters of a patient associated with a radiology work study to assign the radiology work studies to a radiologist work list.

As seen in FIG. 3, a plurality of work lists may be available. For example, each radiologist may be associated with at least one radiologist work list. As such, radiologist work list 370 may be associated with a first radiologist, radiologist work list 380 may be associated with a second radiologist, and radiologist work list 390 may be associated with a third radiologist. The radiologist parameterization filter 360 may assign a radiology work study to the radiologist work lists 370, 380, and/or 390 based on the characteristics, parameters, and/or credentials of the radiologist associated with the radiologist work list 370, 380, and/or 390. In some embodiments, the radiologist parameterization filter 360 may also assign radiology work studies to the radiologist work lists 370, 380, and/or 390 based on parameters of the radiology work studies. As such, the radiologist parameterization filter 360 may assign a first subset of studies (e.g., study 1, study 4, and study 5) to radiologist work list 370, assign a second subset of studies (e.g., study 2 and study 3) to radiologist work list 380, and may assign no studies to the radiologist work list 390. In such a case, the first subset of radiology work studies assigned to radiologist work list 370 may match characteristics, parameters, and/or professional credentials of the radiologist associated with the radiologist work list 370 (e.g., the radiologist associated with the radiologist work list may be better to read or complete the particular radiology work study). Similarly, the second subset of radiology work studies assigned to radiologist work list 380 may match characteristics, parameters, and/or professional credentials of the radiologist associated with the radiologist work list 380. Moreover, as seen in FIG. 3, no radiology work studies were assigned to the radiologist work list 390. For example, none of the radiology work studies may match characteristics, parameters, and/or professional credentials of the radiologist associated with the radiologist work list 390.

In some embodiments, each of the work lists 370, 380, and 390 may be assigned to at least one radiologist. As such, each radiologist work list 370, 380, and 390 may comprise radiology work studies that may be assigned to a radiologist associated with the radiologist work list. In some embodiments, as radiology work studies 310, 320 330, 340, and/or 350 are received, each of the radiology work studies may be assigned one of the radiologist work lists 370, 380, and/or 390. As such, in some embodiments, the radiologist parameterization filter 360 may be used to assign or push radiology work studies to queues or radiologist work lists associated with one or more radiologists.

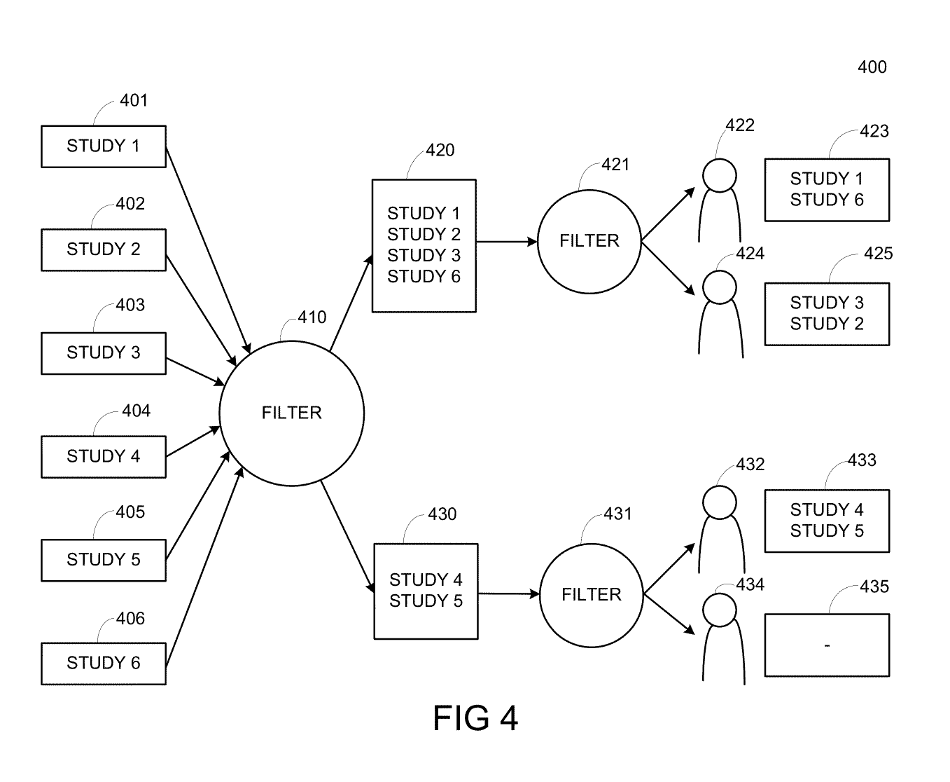

FIG. 4 is a block diagram of a work flow 400 for assigning work studies (e.g., radiology work studies) to be read by one or more medical professionals (e.g., radiologists). In general, the work flow 400 may receive one or radiology more work studies (e.g., radiology work studies 310, 320, 330, 340, and/or 350), filters and/or analyzes the one or more radiology work studies to be assigned to one or more radiologist work lists (e.g., work lists 370, 380, and/or 390), and then assigns the radiology work studies of the one or more radiologists work lists to be read by a radiologist associated with a work list.

As seen in FIG. 4, the work flow 400 may comprise a plurality of radiology work studies 401, 402, 403, 404, 405, and 406. In some embodiments, a filter 410 (e.g., radiologist parameterization filter 360) may receive any or all of the radiology work studies 401, 402, 403, 404, 405, and 406 and assign the radiology work studies to a group radiologist work list 420 and/or 430 (e.g., a sub-specialty radiologist group). For example, the filter 410 may comprise a set of one or more rules, characteristics, and/or parameters as will be discussed in further detail below with regard to FIGS. 6A and 6B. The radiology work studies 401, 402, 403, 404, 405, and 406 may be directed to or assigned to a group radiologist work list 420 and/or 430 based on whether each of the radiology work studies 401, 402, 403, 404, 405, and 406 match or correspond to the rules, characteristics, and/or parameters of the filter 410. For example, radiology work studies 401, 402, 403, and 406 may be assigned to group radiologist work list 420 and radiology work studies 404 and 405 may be assigned to group radiologist work list 430. As such, one or more radiology work studies may be assigned to one or more group radiologist work lists.

As seen in FIG. 4, the radiology work studies (e.g., radiology work studies 401, 402, 403, 404, 405, and/or 406) may be assigned to be read by a radiologist. For example, each of the radiology work studies within the radiology work lists 420 and 430 may be further filtered to be assigned to a radiologist work list associated with one radiologist. In some embodiments, a radiologist work study assignment filter may be used to assign a radiology work study from a group radiologist work list to be read by a radiologist. In the same or alternative embodiments, each group radiologist work list 420 and 430 may correspond to at least one radiologist work study assignment filter. For example, group radiologist work list 420 may be associated with or correspond to radiologist work list assignment filter 421 and group radiologist work list 430 may be associated with or correspond to radiologist work list assignment filter 431. As such, radiologist work list assignment filter 421 may be used to assign the radiology work studies 401, 402, 403, and 406 from the group radiologist work list 420 to a radiologist work list 423 and/or 425. In some embodiments, each radiologist work list may be associated to or correspond with one radiologist. For example, radiologist work list 423 may be associated with or correspond to a radiologist (e.g., a medical professional) 422 and radiologist work list 425 may be associated with or correspond to a radiologist 424. The radiologist work list 423 and 425 may be assigned or populated with radiology work studies from the group radiologist work list 420. For example, after being filtered, the radiology work studies 401 and 406 may be assigned to radiologist work list 423 and radiology work studies 403 and 402 may be assigned radiologist work list 425. Similarly, radiologist work list assignment filter 431 may be used to assign the radiology work studies 404 and 405 from the group radiologist work list 420 to a radiologist work list 433 and/or 435. The radiologist work list 433 may be associated with or correspond to a radiologist 432 and radiologist work list 435 may be associated with or correspond to a radiologist 434. The radiologist work list 434 and 435 may be assigned or populated with radiology work studies from the group radiologist work list 430. For example, after being filtered, the radiology work studies 404 and 405 may be assigned to radiologist work list 433. In such a case, the radiologist work list 435 may not be assigned any radiology work studies.

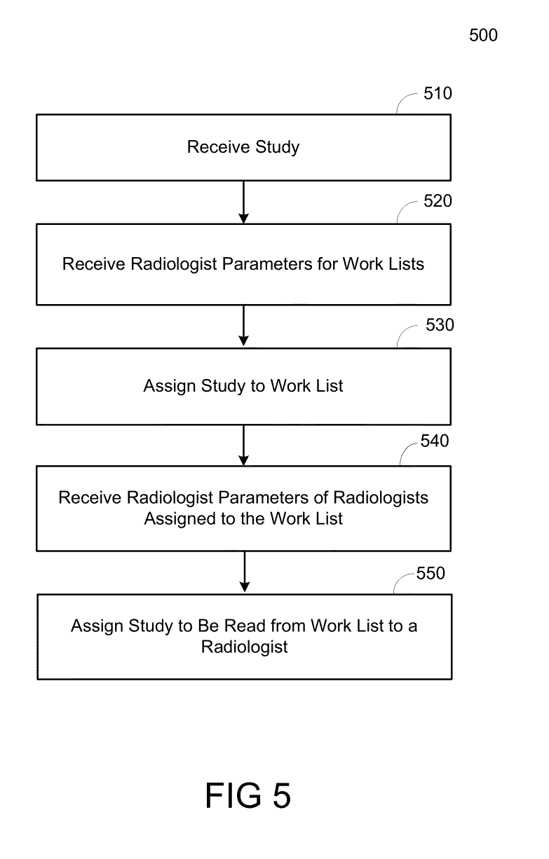

FIG. 5 is a flow diagram of an exemplary method 500 for assigning radiology work studies to a radiologist work list and to assign the radiology work lists to be read by a radiologist. As seen in FIG. 5, at block 510, a work study may be received. For example, a radiology work study (e.g., radiology work study 310, 320, 330, 340, 350, 401, 402, 403, 404, 405, and/or 406) may be received. At block 520, parameters for a radiologist associated with a radiologist work list may be received. For example, radiologist parameters may be received from a filter (e.g., radiologist parameterization filter 360 and/or filter 410). At block 530, the received radiology work study may be assigned to a group radiologist work list. For example, a radiology work study may be assigned to a group radiologist work list (e.g., group radiologist work list 420, and/or 430). At block 540, parameters (e.g., radiologist parameters) of one or more medical professionals assigned to the work list may be received. In some embodiments, the parameters of the medical professional may describe one or more characteristics of the medical professional. Details on parameters that describe one or more characteristics of a medical professional or radiologist are discussed with relation to FIGS. 6A and 6B. In some embodiments, the parameters may be received from or be associated with a filter (e.g., radiologist work list assignment filter 421 and/or 431). At block 550, the received work study may be assigned to a radiologist work list associated with or corresponding to a radiologist (e.g., radiologist work list 423, 425, 433, and/or 435).

As such, a radiology work study may be received. For example, the radiology work study may be a radiology image of a medical patient. The radiology work study may be run through a filter that comprises one or more parameters of radiologists that are used to direct or assign the radiology work study to a group radiologist work list that is created based on the one or more parameters of the radiologists. In some embodiments, each radiologist work list may be associated with or correspond to one or more radiologists. The radiology work study, assigned to the radiologist work list, may be assigned to be read by a radiologist associated with the radiologist work list. For example, each group radiologist work list may correspond to or be associated with a plurality of radiologists where each radiologist is associated with a radiologist work list. The radiology work study from the radiology work list may then be filtered such that the radiology work study is assigned to a radiologist based on the parameters of the radiologists associated with the radiology work list that the radiology work study is assigned to.

FIG. 6A is a diagram illustrating a graphical user interface 600 for setting radiologist parameters for a filter (e.g., radiologist parameterization filter 360, radiology work list 420 and/or 430, and/or filter 421 and/or 431). In general, characteristics, parameters, and/or professional credentials of a radiologist (e.g., medical professionals 161, 162, 163, and/or 170) may be set or configured in a filter. In some embodiments, the filter may then be used to compare the radiologist parameters against characteristics or information associated with radiology work studies (e.g., work studies 310, 320, 330, 340, and/or 350) to create a workflow comprising one or more radiology work lists (e.g., work list 370, 380, 390, 420, and/or 430) and/or assign radiology work studies to a radiologist work list. As such, in some embodiments, the parameters set in a filter may be used to create a radiologist work list with radiology work studies to be assigned to a radiologist based on parameters of the radiologist. In some embodiments, the assigning of the radiology work studies may further be based on parameters of the radiology work studies.

In some embodiments, a plurality of parameters of a radiologist may be set or modified in the filter. For example, parameters may include, but are not limited to, professional credentials of the radiologist (e.g., subspecialty of the radiologist, body part associated with the radiology's expertise, radiologist procedure that the radiologist is specialized in, modality associated with the radiologist's expertise and any other professional credential), a location or jurisdiction associated with the radiologist, imaging viewer or workstation available to the radiologist, the radiologist's work schedule and availability, the radiologist's currently assigned work, the radiologist's current work progress, etc. Further details about each of the above-recited parameters are disclosed below. As such, radiology work lists may be created and radiology work studies may be assigned to a radiologist work list based on a radiologist's parameters.

As seen in FIG. 6A, graphical user interface 600 may comprise a plurality of radiologist parameters (and/or radiology work study parameters) for a particular radiologist work list. The radiologist work list may be assigned to at least one radiologist. A plurality of radiologist parameters (and/or radiology work study parameters) may be used to filter or analyze against radiology work studies such that radiology work studies may be assigned to a radiologist work (e.g., as discussed with relation to FIGS. 3 and 4). In some embodiments, the parameters may further comprise parameters associated with radiology work studies. As such, parameters associated with a radiology work study as well as parameters associated with a radiologist may be used to create a radiology work list or to create an order of radiology work studies to be read within a radiologist work list.

The graphical user interface 600 may comprise a plurality of menus to set parameters of the filter. For example, a radiologist subspecialty menu 610 may be used to select one or more subspecialties of a radiologist to be set in a filter such that the radiologist's subspecialty may be used in the filtering or assigning of radiology work studies. In some embodiments, the radiologist subspecialty may be an expertise or specialty of a radiologist. For example, the radiologist subspecialty may include, but is not limited to, abdomen, neuro/spine, chest, pediatrics, cardiology, and other such medical or radiology specialties. In some embodiments, the graphical user interface 600 may also comprise a menu to select a subspecialty of the procedure or radiology work study. In some embodiments, the subspecialty of the procedure may also be defined in the graphical user interface 600.

In some embodiments, the graphical user interface 600 may comprise a menu may be used to select a human body system that the radiologist may be specialized in. For example, a menu may be used to specify that a radiologist is specialized in radiology work studies associated with the abdomen, neurology and spinal conditions, chest, pediatrics, cardiovascular systems, musculoskeletal systems, urologic, lymphatic, respiratory systems, gastrointestinal, endocrine, and/or reproductive systems, etc. As such, a menu may be used to select human body system that a radiologist may be specialized to read radiology work studies of. In some embodiments, the human body system associated with radiology work study may also be defined in the graphical user interface 600.

In some embodiments, the graphical user interface 600 may comprise a menu 620 to select a region or body part of a patient that the radiologist may be specialized in for reading radiology work studies. Examples of regions or human body parts that the menu 625 may set include, but are not limited to, the head (e.g., skull, brain, eye, ear, neck, orbit, sinus, mastoid, nasal bones), neck, spine, chest (e.g., sternoclavicular joint, sternum, ribs, heart), breast, abdomen, pelvis, hip, and/or extremities (e.g., shoulder, elbow, forearm wrist, hand, femur, knee, tibia, fibula, ankle, foot, etc.). As such, a particular part or general category of the human body of which a radiologist is specialized to read radiology images may be set by menu 620. In some embodiments, the region or body part associated with the radiology work study may also be defined in the graphical user interface 600.

The graphical user interface 600 may further comprise a menu 625 to select a modality (e.g., radiology imaging technique) of which a radiologist may be specialized in. For example, the menu 625 may be used to select particular modalities that the radiologist may be specialized in or should review or read from radiology work studies. Examples of modalities that may be selected may include, but are not limited to, x-ray, computed tomography (CT), magnetic resonance imaging (MRI), radio fluoroscopy, ultrasound, positron emission tomography (PET), and/or nuclear medicine. As such, a type of imaging technology may be associated with a radiologist. In some embodiments, the modality of the radiology work study may also be defined in the graphical user interface 600.

In some embodiments, the graphical user interface 600 may comprise a menu 630 to select a particular procedure for a radiologist. For example, the menu 630 may be used to select a type of radiology procedure that the radiologist may be specialized in. In some embodiments, the procedure may comprise a type of modality and a type of body part or body region of a patient. Procedures may include, but are not limited to, MRI of lower extremities, CT of the spine, CT of a chest, ultrasound of the breast, CT of the head, etc. As such, a procedure may comprise a type of modality, and/or body system, and/or body part, or any other combination of various radiologist parameters. In some embodiments, the procedure associated with the radiology work study may also be defined in the graphical user interface 600.

In the same or alternative embodiments, a priority of the procedure may also be defined by the graphical user interface 600. A patient class associated with the work study may also be defined. For example, the patient class menu 635 may include, but is not limited to, emergency patients, inpatient, and outpatient.

The graphical user interface 600 may further comprise a menu 640 to select or define a location of a radiologist or a jurisdiction or a location that the radiologist may practice or a performing location (e.g., a location where the radiology work study originated from). For example, a radiologist may be licensed or authorized to read radiology work studies from patients in a particular hospital, a state, or any other geographical area. In some embodiments, a particular subspecialty, modality, and/or body part or region may be defined for a particular location. For example, a particular modality (and/or subspecialty and/or body part or region) at a first location or jurisdiction may be defined and another type of modality (and/or subspecialty and/or body part or region) at a second location or jurisdiction may be defined. Thus, particular professional credentials and/or radiologist parameters may be assigned on a per jurisdiction, location, and/or hospital basis. As such, the menu 640 may be used to define locations associated with the radiologist. In some embodiments, the location associated with the work study may also be defined in the graphical user interface 600.

The graphical user interface 600 may further comprise a menu 640 to select one or more imaging viewers (e.g., a type of radiology image viewer such as a type of available picture archiving and communication system) associated with the radiologist. The graphical user interface 600 may further comprise a menu to select a schedule (e.g., available time or work hours) associated with a radiologist.

As such, the graphical user interface 600 may be used to define information (e.g., parameters) associated with a radiologist as well as information associated to a radiology work study. The information set in the graphical user interface 600 may then be used to assign radiology work studies to a particular radiologist work list as discussed in further detail below.

FIG. 6B illustrates a graphical user interface 650 for specifying assignment rules for an assignment filter. In general, one or more rules may be specified for an assignment filter corresponding to a radiologist. As such, the assignment rules specified in the graphical user interface 650 may assign and/or direct one or more radiology work studies to a radiologist work list based on professional parameters of the radiologist.

As seen in FIG. 6B, the graphical user interface 650 comprises an assignment rules list 655. In some embodiments, the assignment rules list 655 may display one or more assignment rules associated with one or more radiologist work lists. As such, the assignment rules list 655 may display one or more assignment rules associated with a radiologist and/or a radiologist work list. The graphical user interface 650 may further comprise an active date column 656 to show when a corresponding assignment rule from the assignment rules list 655 was created and/or how long the corresponding assignment rule has been active for the particular radiologist and/or radiologist work list. Expired date column 657 may specify when an assignment rule from the assignment rules list 655 may expire. As such, the expired date column 657 specifies when an assignment rule will no longer be active for assigning a radiology work study to a radiologist work list. The graphical user interface 650 may also comprise an evaluation sequence column 658 for specifying an evaluation sequence for the assignment rules from the assignment rules list 655. For example, the evaluation sequence may specify an order for executing the assignment rules. As such, in some embodiments, the evaluation sequence may be used to assign when assignment rules may be executed for the purposes of assigning a radiology work study to a radiologist work list.

The graphical user interface 650 may comprise menus to create one or more assignment rules. As seen in FIG. 6B, an activation date selection box 660 may be used to specify a date to activate an assignment rule. For example, the activation date selection box 660 may be used to determine when an assignment rule will begin to function for assigning a radiology work study to a radiologist work list. Similarly, the expiration date selection box 665 may be used to specify date to deactivate an assignment rule. For example, the deactivation date selection box 665 may be used to determine when an assignment rule will no longer function for assigning a radiology work study to a radiologist work list. Furthermore, evaluation sequence selection box 667 may be used to specify an evaluation order or number for an assignment rule, as previously discussed.

As seen in FIG. 6B, the graphical user interface 650 may comprise a study condition menu 670. In some embodiments, the study condition menu 670 may be used to specify conditions for radiology work studies to be assigned. For example, types of radiology work studies that satisfy the conditions set forth in study condition menu 670 may be filtered and/or assigned by an assignment rule if the radiology work study meets the conditions as set forth in study condition menu 670. In some embodiments, the study conditions may comprise whether the radiology work study is associated with information such as a performing organization, procedure subspecialty, patient type, procedure priority, modality, and/or body part. In some embodiments, the graphical user interface 650 may comprise an assignment scheme menu 675 for specifying an assignment scheme for an assignment rule. For example, the assignment scheme may be used to specify an assignment scheme for using the assignment rule to assign radiology work studies. For example, the assignment rule may always be used to assign a radiology work study to a radiologist work list, assign a radiology work study until a capacity of a corresponding radiologist work list is at a certain capacity and then assign the radiology work study to an unassigned work list associated with no radiologist, and/or assign the radiology work study until a radiologist work list is at a certain capacity and then progress to the next valid assignment rule.

The graphical user interface 650 may comprise a specify work list menu 680. In general, the specify work list menu 680 may be used to specify a radiologist work list to which an assignment rule may assign a radiology work list. In some embodiments, a plurality of radiologist work lists may correspond to a single assignment rule. In the same or alternative embodiments, a plurality of parameters may be set for the assignment rule. For example, professional parameters of a radiologist and/or radiology work study parameters, and/or patient parameters may be set in the specify work list menu 680. Such parameters may include, but are not limited to, organization, procedure subspecialty, patient type (e.g., emergency, critical, stat, urgent, routine, etc.), priority (as discussed in further detail below), modality, body part, body system, procedure, procedure modifier, scheduled date and time, completed date and time, performing resource, performing location of the radiology work study, performing imaging archive, requesting physician, requesting location, age of the patient associated with a radiology work study, patient location, and/or study location.

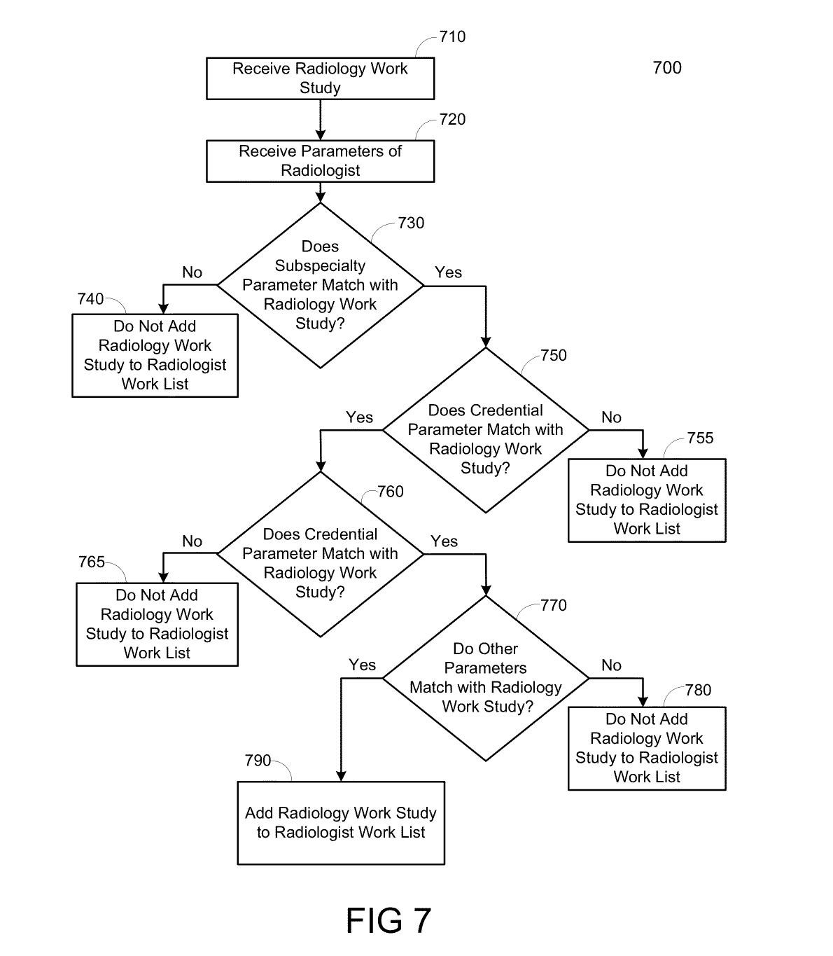

FIG. 7 is a flow diagram of a method 700 for assigning a radiology work study to a radiologist work list. In general, radiology work lists are created based on parameters of a radiologist and a radiology work study may be assigned to the radiology work list if the parameters associated with the radiology work list match or at least partly match with the radiology work study (e.g., the radiologist is determined to be better specialized and/or able to read the radiology work study).

As seen in FIG. 7, at block 710, a radiology work study may be received. For example, the radiology work study may comprise a radiology image of a medical patient. At block 720, parameters of a radiologist are received. The parameters may define one or more characteristics of the radiologist. For example, the parameters may include any or all of the options, parameters, or selections as previously discussed with relation to FIGS. 6A and 6B. At block 730, a decision is made whether a first received parameter of the radiologist associated with the radiology work list matches with the radiology work study. For example, a determination may be made whether the radiologist's subspecialty parameter matches with the radiology work study (e.g., whether the radiologist's defined subspecialty allows the radiologist to read the radiology work study). If the radiologist's subspecialty parameter does not match with the radiology work study, then at block 740 the radiology work study may not be added to the radiologist work list. However, if the radiologist's subspecialty parameter does match with the radiology work study, then at block 750, another determination based on the radiologist's parameters may be made. For example, a determination may be made whether the radiologist's professional credentials parameter matches with the radiology work study. If the radiologist's professional credentials parameter does not match with the radiology work study, then at block 755, the radiology work study may not be added to the radiologist work list. However, if the radiologist's professional credentials parameter does match with the radiology work study, then at block 760, another determination may be made based on the radiologist's parameters. For example, a determination may be made whether the radiologist's location or jurisdictions parameter matches with the radiology work study. If the radiologist's location or jurisdictions parameter does not match with the radiology work study, then at block 765, the radiology work study may not be added to the radiologist work list. However, if the radiologist's location or jurisdictions parameter does match with the radiology work study, then at block 770, another determination may be made based on the radiologist's parameters. For example, a determination may be made based on any of the parameters, settings, selections, and/or characteristics previously discussed with relation to FIGS. 6A and 6B. If the radiologist's additional parameter does not match with the radiology work study, then at block 780, the radiology work study may not be added to the radiologist work list. However, if the radiologist's additional parameter does match with the radiology work study, then at block 790, then the radiology work study may be added to the radiologist work list.

As such, a radiology work study may be assigned to a radiologist work list based on the subspecialty of the radiologist, credentials of the radiologist, location or jurisdiction, of the radiologist, or any other characteristic of the radiologist.

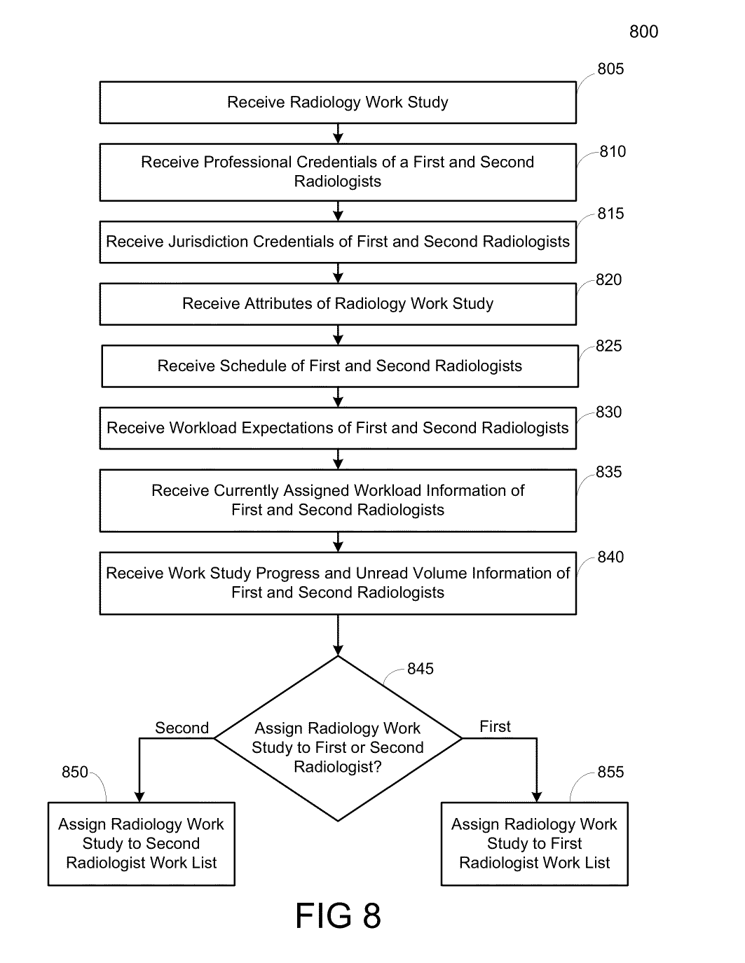

FIG. 8 illustrates an example method 800 for a radiology workflow to assign a radiology work study to either a first radiologist work list or a second radiologist work list. In general, the method 800 may receive a plurality of parameters of a first radiologist and a second radiologist and assign a radiology work study to either the first radiologist or the second radiologist by assigning the radiology work study to a radiologist work list.

As seen in FIG. 8, at block 805, a radiology work study may be received. In some embodiments, the radiology work study may comprise a radiology image to be read by a radiologist. At block 810, professional credentials of a first radiologist and a second radiologist may be received. In some embodiments, professional credentials of each of a plurality of radiologists may be received. The professional credentials may comprise, but are not limited to, a radiologist's subspecialty, a modality of which the radiologist may specialize in, a type of procedure that the radiologist may specialize in, and/or any of the parameters or characteristics as previously discussed with regard to FIGS. 6A and 6B.

At block 815, jurisdiction credentials of a first radiologist and a second radiologist may be received. For example, particular hospitals, states, and other locations for which each of the radiologists may have particular credentials may be received. Jurisdiction credentials may comprise, but are not limited to, a radiologist having credentials to read a particular type of radiology study from a particular jurisdiction (e.g., a certain modality from a radiology image of a patient who is located in a particular jurisdiction, reading a particular body system from a radiology image of a patient who is located in another jurisdiction, etc.). As such, in some embodiments, jurisdiction credentials may comprise an indication whether a particular radiologist has a credential to read a particular radiology image corresponding to a patient at a particular jurisdiction.

At block 820, attributes of the radiology work study may be received. For example, meta attributes of a radiology work study may be received. In some embodiments, attributes of a work study may include, but are not limited to, any of the characteristics, credentials, and parameters as previously discussed with relation to FIGS. 6A and 6B.

At block 825, a schedule of a first radiologist and a second radiologist may be received. For example, a work schedule of radiologists may be received. In some embodiments, the work schedule may provide an indication as to whether a particular radiologist is currently reading and/or actively completing radiology work studies that have been assigned to the radiologist work list associated with the radiologist. As such, scheduled work hours of radiologists may be received. At block 830, workload expectations of radiologists may be received. For example, workload expectations of a first radiologist and a second radiologist may be received. In some embodiments, workload expectations may comprise an indication as to a number of radiology work studies a radiologist is expected to read and/or complete within a particular time. In some embodiments, the workload expectation may comprise a relative value unit (RVU) that a radiologist may be expected to complete within a particular time period. In some embodiments, each type of radiology work study may correspond to a particular RVU. For example, a chest x-ray may correspond to 1 RVU and a chest CT may correspond to 20 RVUs. As such, the RVU corresponding to various types of radiology work studies may vary. In some embodiments, the workload expectations of the radiologists may comprise a workload expectation relative to a radiologist's specialty (e.g., a particular parameter associated with the radiologist). For example, the workload expectation may comprise a number of radiology work studies and/or RVUs of radiology work studies within a radiologist's specialty that the radiologist is expected to read and/or complete.

At block 835, currently assigned workload information may be received. For example, the currently assigned workload for a first radiologist and a second radiologist may be received. In some embodiments, the assigned workload may comprise a number of radiology work studies that have been assigned to a radiologist (e.g., to a radiologist work list associated with the radiologist) and/or a number of RVUs associated with the radiology work studies that have been assigned to the radiologist. In some embodiments, currently assigned radiology work studies may comprise unread radiology work studies and/or radiology work studies that have been read and/or completed by the radiologist. At block 840, radiology work study progress and/or unread radiology work study information may be received. For example, the work progress of a first radiologist and a second radiologist may be received. In some embodiments, the work progress of radiology work studies within a radiologist's specialty and the work progress of radiology work studies outside of a radiologist's specialty may be received. As such, in some embodiments, a rate of progress (e.g., a rate at which a radiologist is reading and/or completing a radiology work study) may be received.

At block 845, a determination may be made as to whether to assign the radiology work study to either the radiologist work list associated with a first radiologist or the radiologist work list associated with the second radiologist. In some embodiments, any or all of the previously discussed parameters or information (e.g., at blocks 810, 815, 820, 825, 830, 835, 840, and/or parameters as discussed with regard to FIGS. 6A and 6B) may be used to determine which radiologist work list to assign the radiology work study. In some embodiments, the radiology work study may be assigned to a radiologist who most matches with the radiology work study (e.g., the radiologist who most closely matches with the attributes of the radiology work study, a radiologist with certain credentials and parameters who may be best suited to read the radiology image study at a particular time from a particular jurisdiction, etc.). As such, if a determination is made that the second radiologist should read the radiology work study, then at block 850, the radiology work study may be assigned to a second radiologist work list associated with the second radiologist. However, if a determination is made that the first radiologist should read the radiology work study, then at block 855, the radiology work study may be assigned to a first radiologist work list associated with the first radiologist.

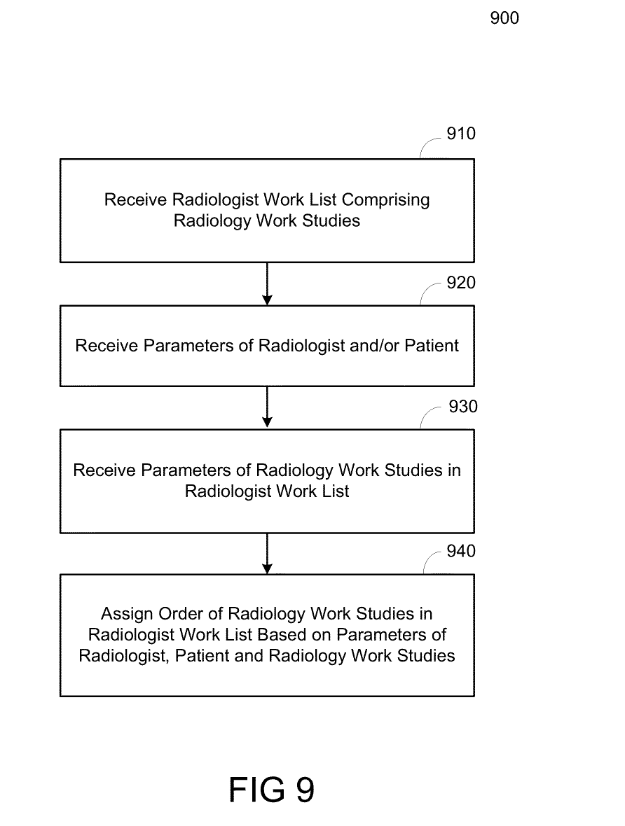

FIG. 9 illustrates a flow diagram of a method 900 for assigning or ordering radiology work studies within a radiologist work list. In general, the method 900 may assign or order radiology work studies in a radiologist work list (e.g., radiologist work list 370, 380, 390, 423, 425, 433, and/or 435) to be read by a radiologist. As such, the method 900 may comprise a push approach for assigning or determining an ordering of radiology work studies for a radiologist to read or complete.

As seen in FIG. 9, at block 910, a radiologist work list comprising radiology work studies may be received. At block 920, parameters associated with the radiologist associated with the radiology work list and/or patients associated with the radiology work studies may be received. For example, the parameters may comprise the subspecialty, credentials, location, or any other parameter or characteristic of the radiologist as previously discussed with relation to FIGS. 6A and 6B and/or elsewhere within the present disclosure. Parameters of a patient may comprise any information of a patient as discussed within the present disclosure. For example, the parameters of a patient may comprise a patient's age, location, type (e.g., emergency patient, inpatient, outpatient, etc.). As such, any parameters used for assigning a radiology work study may also be used to determine an order for radiology work studies within a radiologist work list. At block 930, the parameters of the radiology work studies may be received. For example, the parameters of radiology work studies in the radiologist work list may be received. In some embodiments, the parameters of the radiology work study comprise any parameter or characteristic as previously discussed with relation to FIGS. 6A and 6B and/or elsewhere within the present disclosure. For example, the parameters of the radiology work study may comprise a service level priority associated with the radiology work study. At block 940, the order of the radiology work studies may be assigned based on the parameters of the radiologist and/or the parameters of the radiology work studies, and/or parameters of the patient associated with the radiology work study.

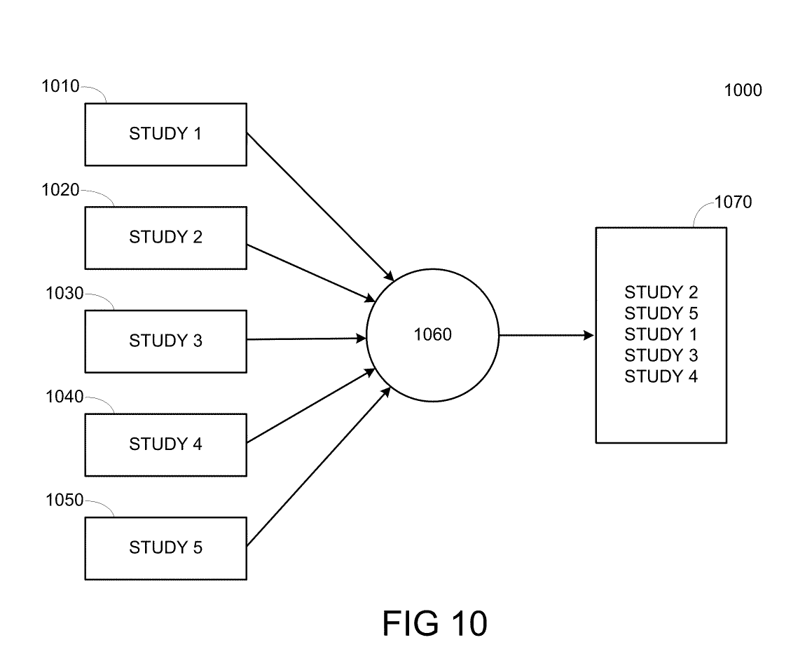

FIG. 10 is an example workflow 1000 of assigning an order of work studies within a work list. In general, radiology work studies may be assigned an order to be read within a radiologist work list. In some embodiments, the workflow 1000 uses the method 900 as disclosed with regard to FIG. 9.

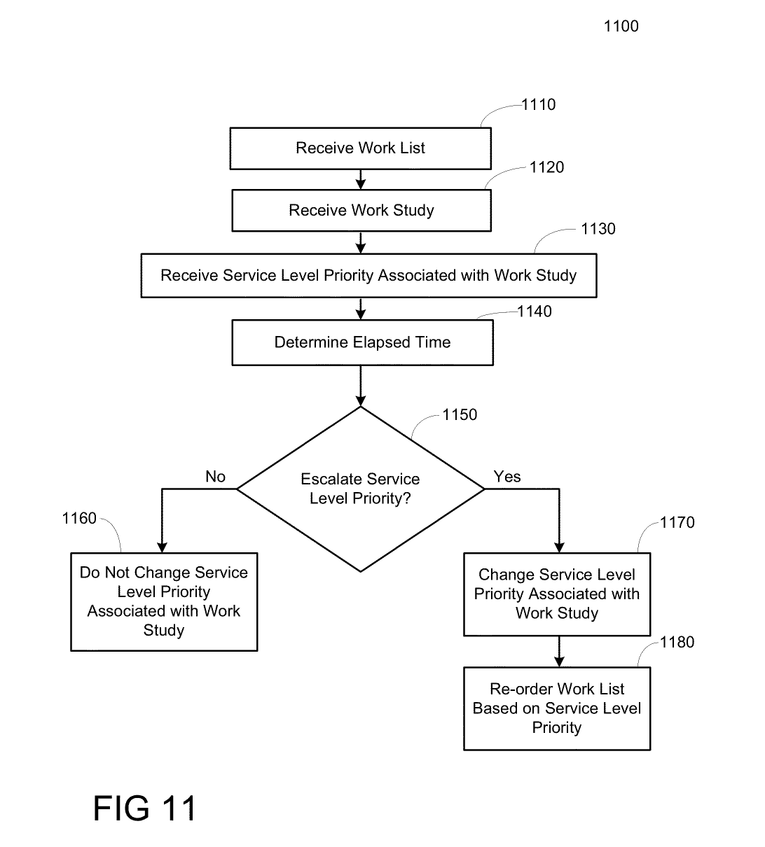

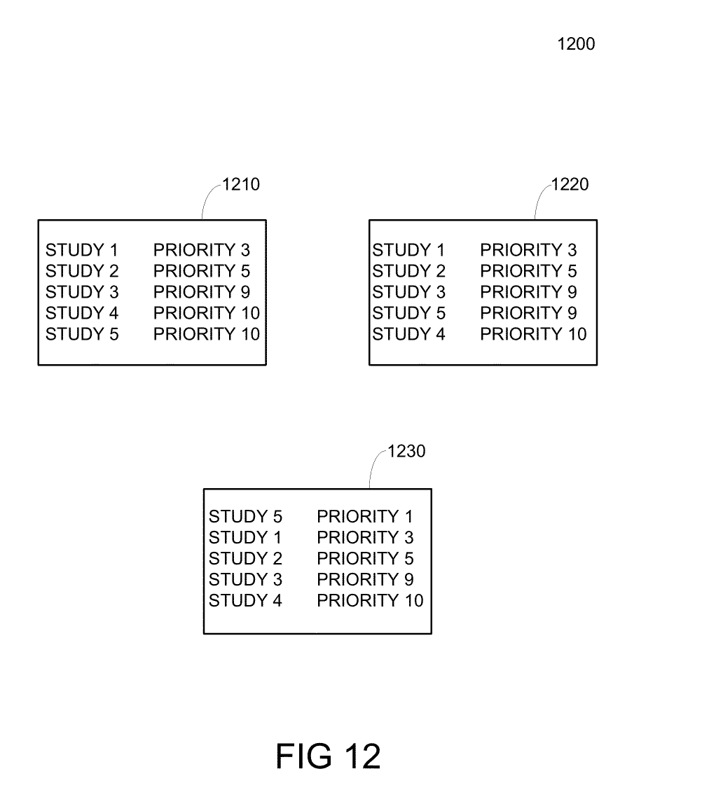

As seen in FIG. 10, the example workflow 1000 comprises radiology work studies 1010, 1020, 1030, 1040, and/or 1050. Each of the radiology work studies may be associated with one radiologist work list. In some embodiments, the radiology work studies 1010, 1020, 1030, 1040, and/or 1050 may be assigned an order to be read by a radiologist. In the same or alternative embodiments, the radiology work studies may be assigned an order to be read by the radiologist associated with the radiologist work list. The order of the radiology work studies may be based on parameters of the radiologist and/or parameters of the radiology work studies. For example, the order of the radiology work studies may be at least partly based on a service level priority associated with the radiology work studies as discussed in further detail below with regard to FIGS. 11 and 12. As such, the radiology work studies may be processed and/or analyzed by a filter or radiology work study priority module 1060 to determine or assign an order for the radiology work studies to be read within a radiologist work list. As such, radiology studies 1010, 1020, 1030, 1040, and/or 1050 may be arranged or ordered within a radiologist work list 1070 such that radiology work study 2 has a higher service level priority than radiology work study 5. Similarly, radiology work study 1 may be assigned or placed in an order such that radiology work study 1 comprises a lower service level priority than radiology work study 5, radiology work study 3 may be assigned or placed in an order such that radiology work study 3 comprises a lower service level priority than radiology work study 1, and radiology work study 4 may be assigned or placed in an order such that radiology work study 4 comprises a lower service level priority than radiology work study 3. In some embodiments, the order of the radiology work studies within the radiologist work list 1070 may comprise an order that the radiologist associated with the radiologist work list 1070 must read the radiology work studies. In the same or alternative embodiments, a higher priority may comprise an indication that the radiologist associated with the radiologist work list 1070 should read the radiology work study with the higher service level priority before reading a radiology work study with a lower service level priority.

FIG. 11 illustrates an example method 1100 for auto escalating a service level priority associated with a work study. In general, the method 1100 changes a service level priority associated with a work study (e.g., a radiology work study) and may reorder or reassign the work studies within a work list (e.g., a radiologist work list) based on the service level priority of the work studies.

As seen in FIG. 11, at block 1110, a work list may be received. For example, a radiologist work list may be received. In some embodiments, the received radiologist work list may comprise a plurality of radiology work studies. At block 1120, one of the work studies of the work list are received. For example, a radiology work study from the radiologist work study may be selected and/or received. At block 1130, a parameter of the radiology work study may be received. For example, a service level priority associated with the radiology work study may be received. In some embodiments, the service level priority may be associated with a service level agreement. In the same or alternative embodiment, the service level agreement may specify an amount of time that a particular radiology work study will be read and/or completed by a radiologist. For example, a service level agreement may comprise a contractual obligation that a particular type of radiology work study (e.g., radiology work studies associated with a patient type parameter of ‘emergency’) will be completed (e.g., read by a radiologist) within a predefined period of time.

At block 1140, an elapsed time for the radiology work study may be determined. For example, the radiology work study may have been assigned to the radiologist work list received at block 1110 at a first time interval. At block 1140, the time that has progressed from the first time interval may be determined. In some embodiments, at block 1150, a determination may be made whether to escalate the service level priority of the radiology work study. In the same or alternative embodiments, the decision to escalate the service level priority of the radiology work study may be based on the current service level priority of the radiology work study and/or the elapsed time associated with the radiology work study. For example, the service level priority may be specified as a number between 1 and 10 where a service level priority of 1 may comprise a higher service level priority than a service level priority of 10 (e.g., the radiology work study with a service level priority of 1 may need to be read by the radiologist before the radiologist reads the radiology work study with a service level priority of 10). In some embodiments, the service level priority assigned to a radiology work study may be based on the service level agreement associated with the radiology work study and/or the elapsed time associated with the radiology work study. For example, if a radiology work study is associated with a 30 minute service level agreement time, then every three minutes (e.g., each service level priority number from 1 through 10 may indicate 10% of the service level agreement time) of elapsed time associated with the radiology work study may comprise a separate service level priority level. As such, if the radiology work study has an elapsed time of 3 or less minutes then the service level priority for the radiology work study may be 10, if the radiology work study has an elapsed time of 6 or less minutes then the service level priority for the radiology work study may be 9, and so forth until a time when the radiology work study has an elapsed time of 3 or less minutes, at which point the service level priority of the radiology work study may be escalated to a value of 1.

As seen in FIG. 11, if the service level priority of the radiology work study does not need to be escalated, then at block 1160, the service level priority associated with the radiology work study is not changed. However, if the service level priority of the radiology work study does need to be escalated, then at block 1170, the service level priority associated with the radiology work study may be changed. At block 1180, the work list with the work studies may be reordered. For example, the radiology work studies of a radiologist work list may be reordered or reassigned based on the service level priorities of the radiology work studies. As such, if a service level priority of a radiology work study is escalated (e.g., changed), then the order of the radiology work study within the radiologist work list that comprises additional radiology work studies may be changed or ordered differently. Thus, a particular radiology work study may at first be at the bottom of a radiologist work list, but as the elapsed time associated with the radiology work study progresses, the radiology work study may increase in service level priority and be reordered ahead or above other radiology work studies or reassigned within the radiologist work list based on the change in the service level priority.

FIG. 12 is an example of a work study with an escalated service level priority and being reordered or reassigned within a work list. In general, a radiology work study may comprise or be associated with a first service level priority and after an elapsed amount of time the radiology work study may comprise or be associated with a second service level priority. In some embodiments, the change in service level priority may result in a change of the ordering or assigning of the radiology work study within a radiologist work list.

As seen in FIG. 12, a radiologist work list 1210 represents a particular radiologist work list at a first time interval. The radiologist work list 1210 may comprise a plurality of radiology work studies such as study 1 with a service level priority of 3, study 2 with a service level priority of 5, study 3 with a service level priority of 9, study 4 with a service level priority of 10, and study 5 with a service level priority of 10. As such, the order or assignment of the radiology work studies within the radiologist work list 1210 at a first time interval may be based on the service level priority of the radiology work studies. Radiologist work list 1220 may represent the radiologist work list 1210 at a second time interval. For example, the radiologist work list 1220 may represent the radiologist work list 1210 after an elapsed period of time. In some embodiments, the elapsed period of time may result in a change of service level priorities associated with a radiology work study. For example, study 5 may be associated with an escalated service level priority. As such, the service level priority of study 5 may be changed from 10 to 9 and the change in the service level priority may result in a reordering or reassignment of study 5 within its radiologist work list. For example, study 5 may be reordered or reassigned within the radiologist work list 1220 to be placed above study 5 within the radiologist work list 1220 Similarly, radiologist work list 1230 represents the radiologist work list 1210 at a third time interval. For example, the radiologist work list 1230 may represent the radiologist work list 1220 (or 1210) after a second elapsed period of time. As seen, the service level priority associated with study 5 has been escalated such that study 5 has now been reordered or reassigned to the top of the radiologist work list.

In some embodiments, a service level priority as disclosed herein may be based on a service level time (as discussed above) and/or a clinical priority associated with the radiology work study. For example, a service level priority may be based on a turnaround time for the radiology work study and a clinical priority associated with the radiology work study. In some embodiments, the clinical priority may be based on a patient's age (e.g., an older patient may comprise a higher priority than a younger patient), patient type (e.g., emergency, critical, stat, urgent, routine), and/or other parameters associated with the patient. As such, a service level priority may be based on the service turnaround time associated with the radiology work study as well as a clinical priority associated with a patient corresponding to the radiology work study. Moreover, in some embodiments, the escalation of a service level priority (as discussed above with regard to FIG. 11) may be based both on the service turnaround time (e.g., elapsed time) and a clinical priority (e.g., a change in a patient type to emergency and/or urgent may further escalate the service level priority).

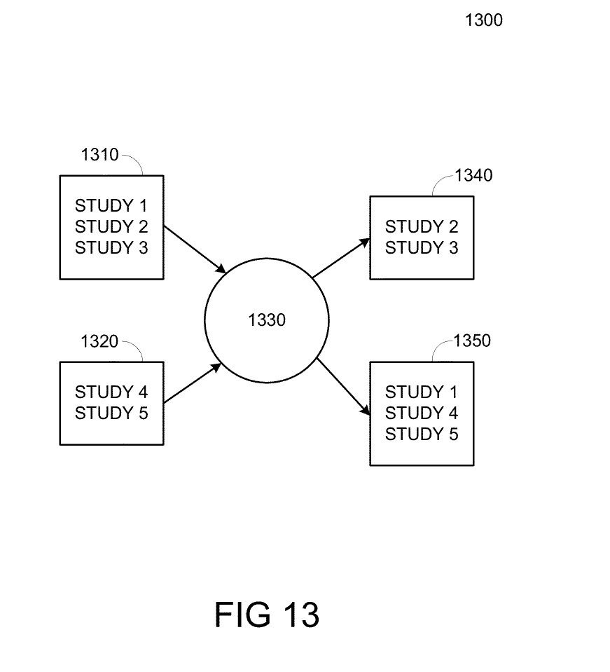

FIG. 13 illustrates an example 1300 of reassigning a work study from a first work list to a second work list. In general, a radiology work study may be reassigned from a first radiologist work list to a second radiologist work list. The reassigning of the work study may be based on a service level agreement (e.g., service level priority values) and/or the rate at which the radiologist associated with the first radiologist work list is progressing through, reading, and/or completing the radiology work studies in the first radiologist work list of which the radiologist is associated.

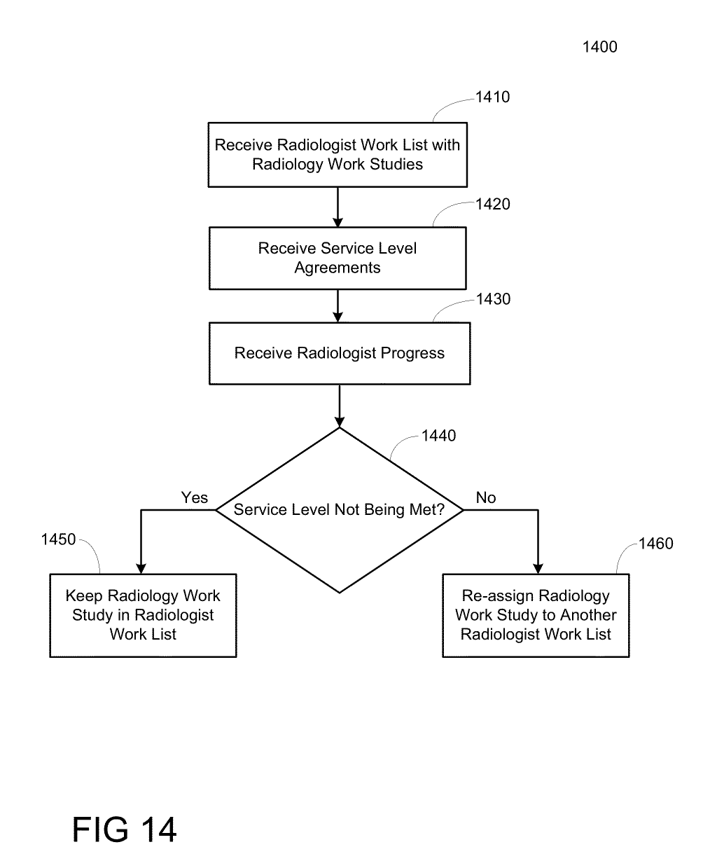

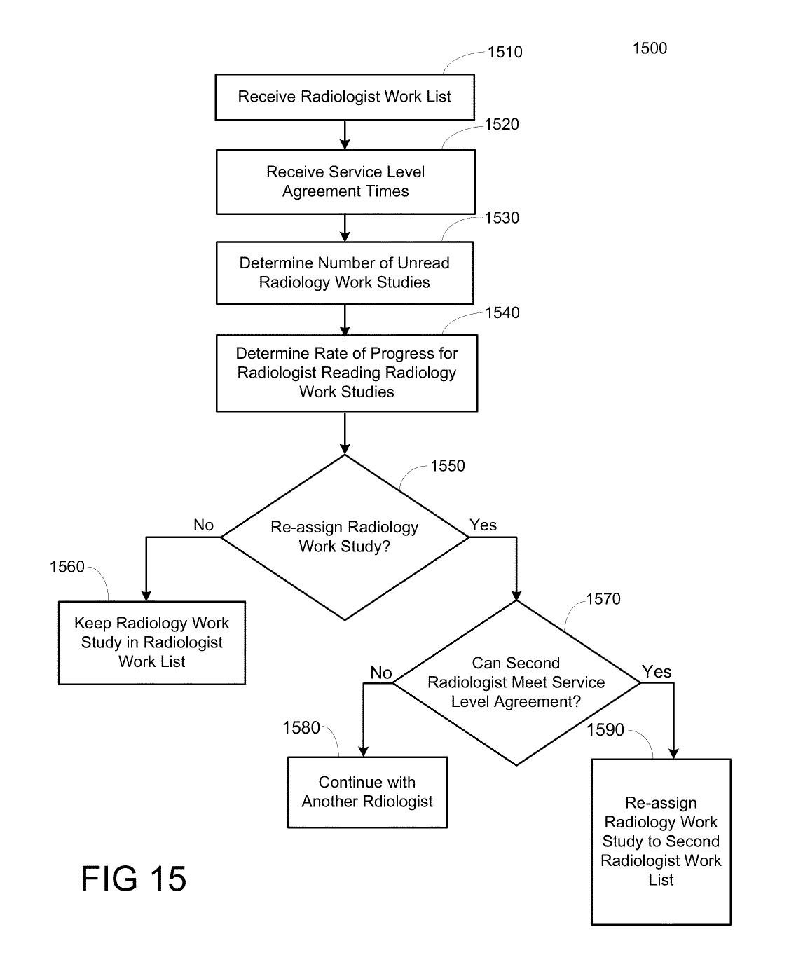

As seen in FIG. 13, the example 1300 may comprise a first radiologist work list 1310 and a second radiologist work list 1320. In some embodiments, the first radiologist work list 1310 and the second radiologist work list 1320 may each comprise one or more radiology work studies. For example, the first radiologist work list may comprise study 1, study 2, and study 3 and the second radiologist work list 1320 may comprise study 4 and study 5. In some embodiments, a filter or module 1330 (e.g., a module performing the method 1400 of FIG. 14 and/or the method 1500 of FIG. 15) may analyze the first radiologist work list and the second radiologist work list and/or the radiology work studies within each of the radiologist work lists. For example, the module 1330 may determine that a radiology work study should be reassigned from the first radiologist work list to the second radiologist work list. As such, study 1 may be reassigned from the first radiologist work list to the second radiologist work list, as seen in updated radiologist work list 1340 and updated radiologist work list 1350. Further details with regard to determining whether to reassign a radiology work study from a first radiologist work list to a second radiologist work list are described in further detail below with regard to FIGS. 14 and 15.

FIG. 14 illustrates an example method 1400 for reassigning a work study to another work list. In general, the method 1400 may receive reassign a work study to another work list based on service level agreements and a rate of progress of reading and/or completing work studies.

As seen in FIG. 14, at block 1410, a work list with work studies may be received. For example, a radiologist work list with radiology work studies may be received. In some embodiments, the radiologist work list may be associated with a radiologist. At block 1420, service level agreements may be received. In some embodiments, a service level agreement for each of the radiology work studies may be received. In the same or alternative embodiment, the information with regard to service level agreements may be received. For example, an elapsed time of a radiology work study may be received (e.g., the amount of time that has elapsed since the radiology work study has been assigned to the radiologist work list) and/or a time left in order to meet a service level agreement time may be received. At block 1430, radiologist progress information may be received. For example, the rate at which a radiologist has been reading or completing radiology work studies and/or an unread radiology work study volume of the radiologist may be received.

At block 1440, a determination may be made as to whether a service level agreement may or may not be met. In some embodiments, the determination may be based on the received service level agreements and the rate of progress of the radiologist associated with the radiologist work list. If the service level agreement time is expected to be met (e.g., the radiologist is expected to read and/or complete the radiology work study before the elapsed time of the radiology work study exceeds the service level agreement time), then at block 1450, the radiology work study may remain in the radiologist work list. However, if the service level agree time is not expected to be met (e.g., the radiologist is not expected to read and/or complete the radiology work study before the elapsed time of the radiology work study exceeds the service level agreement time), then at block 1460, the radiology work study may be assigned to another radiologist work list associated with another radiologist.

FIG. 15 illustrates an example method 1500 for determining whether to keep a radiology work study in a radiologist work list or to reassign the radiology work study from the radiologist work list to a second radiologist work list.

As seen in FIG. 15, at block 1510, a radiologist work list may be received. In some embodiments, the radiologist work list may comprise a plurality of radiology work studies. In the same or alternative embodiments, the radiologist work list may be associated with one radiologist. At block 1520, service level agreement times may be received. For example, the service level agreement times may comprise a maximum amount of time allowed to elapse when the radiology work study has entered into the workflow and/or an amount of time remaining before the service level agreement time expires (e.g., the elapsed time of the radiology work study has exceeded the maximum amount of time of the service level agreement associated with the radiology work study). In some embodiments, the service level agreement times for each of the radiology work studies in the radiologist work list may be received. At block 1530, a number of unread radiology work studies in the radiologist work list may be received. In some embodiments, each unread radiology work study may comprise a radiology work study that has not been completed or read by the radiologist. At block 1540, a rate of progress (e.g., rate at which the radiologist is reading and/or completing radiology work studies assigned to the radiologist work list) may be determined and/or received.

At block 1550, a determination is made as to whether to reassign the radiology work study. In some embodiments, the reassigning of the radiology work study may be based on the service level agreement times and the rate of progress of the radiologist. If a determination is made to not reassign the radiology work study, then at block 1560, the radiology work study may be kept or remain in the radiologist work list. However, if a determination is made to reassign the radiology work study (e.g., the service level agreement time is not expected to be met), then at block 1570, a determination may be made as to whether a second radiologist may meet or is expected to meet the service level agreement time associated with the radiology work study. In some embodiments, the determination may be based on the service level agreement times of radiology work studies in the second radiologist work list associated with the second radiologist as well as a rate of progress of the second radiologist. In some embodiments, parameters or characteristics of the second radiologist may be used to determine if the radiology work study may be assigned to the second radiologist. If it is determined that the second radiologist may or is expected to meet the service level agreement time of the radiology work study, then at block 1590, the radiology work study may be reassigned from the first radiologist work list to the second radiologist work list. In some embodiments, reassigning a radiology work study from the first radiologist work list to the second radiologist work list may comprise removing the radiology work study from the first radiologist work list and adding the radiology work study to the second radiologist work list. However, if it is determined that the second radiologist is not expected to meet the service level agreement time and/or cannot read the radiology work study, then at block 1580, the method may continue with finding another radiologist who may read the radiology work study and meet the service level agreement time.

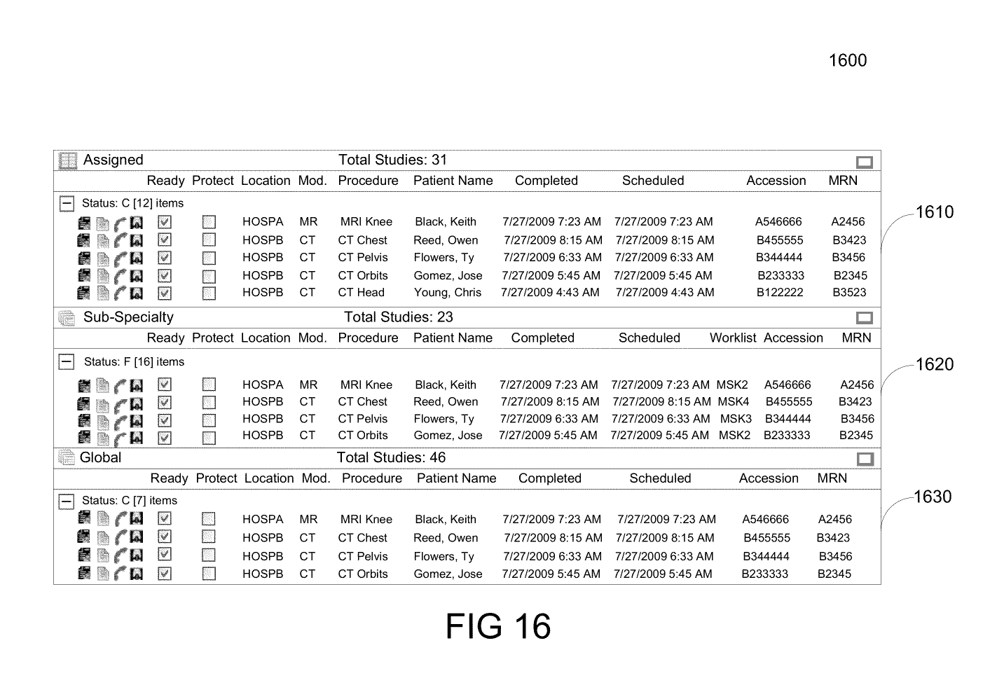

FIG. 16 is a graphical user interface 1600 comprising a plurality of work lists. In general, the graphical user interface 1600 may display an assigned work list and one or more filtered work lists.

As seen in FIG. 16, the graphical user interface 1600 may comprise an assigned work list 1610. In some embodiments, the assigned work list 1610 may comprise work studies assigned to a medical professional. For example, the assigned work list 1610 may comprise radiology work studies that have been assigned to a radiologist. In some embodiments, the radiology work studies in the assigned work list 1610 may comprise radiology work studies that must be read by the radiologist associated with the assigned work list 1610. In the same or alternative embodiments, the order of the radiology work studies in the assigned work list may be based on parameters of the radiologist and/or parameters of the radiology work studies. As such, the assigned work list 1610 may comprise a push approach where radiology work studies are assigned to a radiologist.

The graphical user interface 1600 may further comprise a filtered work list 1620. In some embodiments, the filtered work list 1620 may filter available work studies based on parameters of the radiologist. For example, the filtered work list 1620 may comprise a subspecialty work list where radiology work studies that are similar to or match with the radiologist's subspecialty parameter may be viewed by the radiologist. As such, the filtered work list 1620 may comprise a work list of radiology work studies relating to a work group to which the radiologist is associated. Furthermore, as seen in FIG. 16, the graphical user interface 1600 may further comprise a work list 1630. In some embodiments, the work list 1630 may comprise work studies from an entire organization. For example, the work list 1630 may comprise a list of all available radiology work studies. In some embodiments, the radiologist (e.g., the radiologist associated with the assigned work list 1610) may select and/or choose radiology work studies from the filtered list 1620 and/or the work list 1630. In the same or alternative embodiments, such selected radiology work studies may then be placed into the assigned work list 1610 associated with the radiologist. Furthermore, the assigned work list 1610 may further sort or order the radiology work studies in the assigned work list 1610 in order of priority. In some embodiments, the priority of a radiology work study may indicate an order that the radiologist must read the radiology work study or a time frame that the radiologist needs to read the radiology work study in the assigned work list 1610.

As such, the graphical user interface 1600 may comprise one or more work lists. In some embodiments, the graphical user interface 1600 may comprise a work list of radiology work studies that have been assigned to a specific radiologist. The graphical user interface 1600 may further comprise a work list of radiology work studies that the radiologist may work on due to relating to or matching with a parameter of the radiologist, but have yet to be assigned to the radiologist or any other radiologist. In some embodiments, the graphical user interface 1600 may further comprise a work list of all radiology work studies.

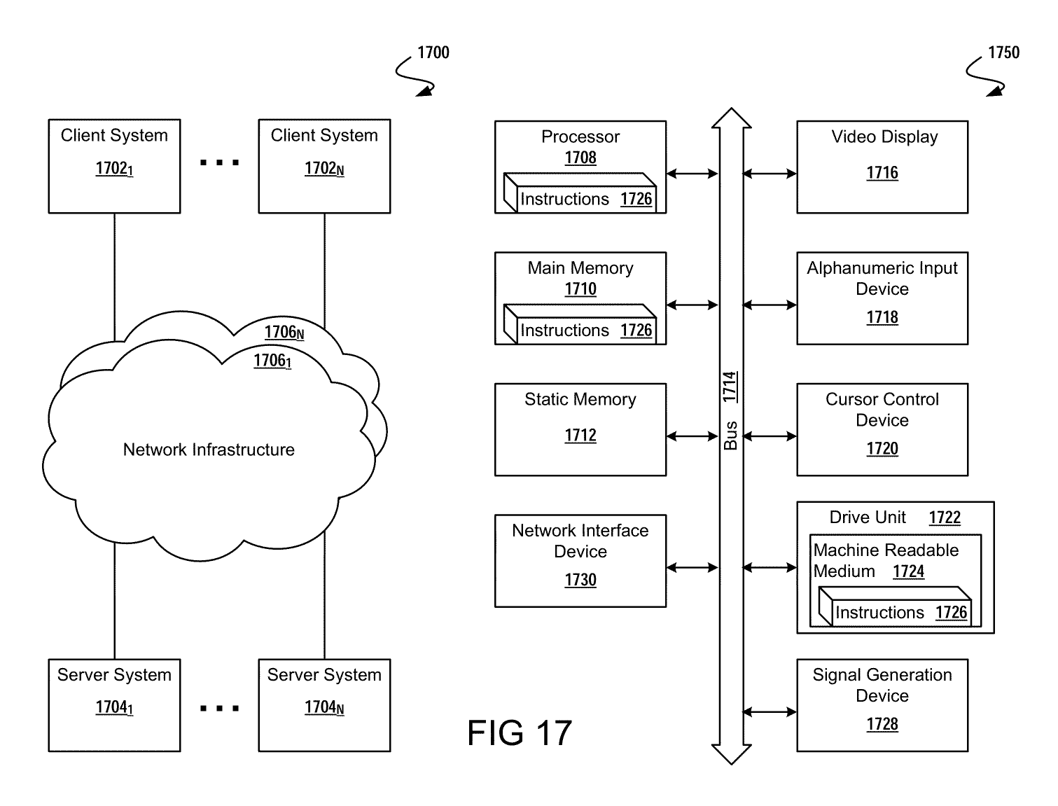

FIG. 17 depicts a diagram illustrating an exemplary computing system environment for execution of the operations comprising various embodiments of the disclosure. In some embodiments, a network 1700, including nodes for client computer systems 1702₁ through 1702_{N}, nodes for server computer systems 1704₁ through 1704_{N}, nodes for network infrastructure 1706₁ through 1706_{N}, any of which nodes may comprise a machine 1750 within which a set of instructions for causing the machine to perform any one of the techniques discussed above may be executed. The embodiment shown is purely exemplary, and might be implemented in the context of one or more of the figures herein.

Any node of the network 1700 may comprise a general-purpose processor, a digital signal processor (DSP), an application specific integrated circuit (ASIC), a field programmable gate array (FPGA) or other programmable logic device, discrete gate or transistor logic, discrete hardware components, or any combination thereof capable to perform the functions described herein. A general-purpose processor may be a microprocessor, but in the alternative, the processor may be any conventional processor, controller, microcontroller, or state machine. A processor may also be implemented as a combination of computing devices (e.g. a combination of a DSP and a microprocessor, a plurality of microprocessors, one or more microprocessors in conjunction with a DSP core, or any other such configuration, etc.).

In alternative embodiments, a node may comprise a machine in the form of a virtual machine (VM), a virtual server, a virtual client, a virtual desktop, a virtual volume, a network router, a network switch, a network bridge, a personal digital assistant (PDA), a cellular telephone, a web appliance, or any machine capable of executing a sequence of instructions that specify actions to be taken by that machine. Any node of the network may communicate cooperatively with another node on the network. In some embodiments, any node of the network may communicate cooperatively with every other node of the network. Further, any node or group of nodes on the network may comprise one or more computer systems (e.g. a client computer system, a server computer system) and/or may comprise one or more embedded computer systems, a massively parallel computer system, and/or a cloud computer system.

The computer system 1750 includes a processor 1708 (e.g. a processor core, a microprocessor, a computing device, etc.), a main memory 1710 and a static memory 1712, which communicate with each other via a bus 1714. The machine 1750 may further include a display unit 1716 that may comprise a touch-screen, or a liquid crystal display (LCD), or a light emitting diode (LED) display, or a cathode ray tube (CRT). As shown, the computer system 1750 also includes a human input/output (I/O) device 1718 (e.g., a keyboard, an alphanumeric keypad, etc.), a pointing device 1720 (e.g., a mouse, a touch screen, etc.), a drive unit 1722 (e.g. a disk drive unit, a CD/DVD drive, a tangible computer readable removable media drive, an SSD storage device, etc.), a signal generation device 1728 (e.g. a speaker, an audio output, etc.), and a network interface device 1730 (e.g. an Ethernet interface, a wired network interface, a wireless network interface, a propagated signal interface, etc.).

The drive unit 1722 includes a machine-readable medium 1724 on which is stored a set of instructions (i.e. software, firmware, middleware, etc.) 1726 embodying any one, or all, of the methodologies described above. The set of instructions 1726 is also shown to reside, completely or at least partially, within the main memory 1710 and/or within the processor 1708. The set of instructions 1726 may further be transmitted or received via the network interface device 1730 over the network bus 1714.

It is to be understood that embodiments of this disclosure may be used as, or to support, a set of instructions executed upon some form of processing core (such as the CPU of a computer) or otherwise implemented or realized upon or within a machine- or computer-readable medium. A machine-readable medium includes any mechanism for storing information in a form readable by a machine (e.g. a computer). For example, a machine-readable medium includes read-only memory (ROM); random access memory (RAM); magnetic disk storage media; optical storage media; flash memory devices; electrical, optical or acoustical or any other type of media suitable for storing information.

What is claimed is:

1. A method for assigning work studies to be read within a work list, the method comprising:
receiving a radiologist work list comprising a plurality of radiology work studies;
receiving at least one parameter associated with the radiology work studies in the radiologist work list, the at least one parameter comprising a service level priority for the radiology work studies; and
assigning, by a computer, an order for the radiology work studies in the radiologist work list based on the at least one parameter associated with the radiology work studies.

2. The method of claim 1, wherein the order of the radiology work studies in the radiologist work list is assigned such that the order specifies a priority for the radiology work studies to be read by a radiologist associated with the radiologist work list.

3. The method of claim 1, wherein the ordering of the radiology work studies is further based on a parameter of the radiologist comprising at least one of a subspecialty of the radiologist, a professional credential of the radiologist, and a location of the radiologist.

4. The method of claim 1, wherein the service level priority associated with each of the radiology work studies is updated based on an elapsed time associated with each of the radiology work studies.

5. The method of claim 4, wherein an updating of the service level priority associated with each of the radiology work studies comprises a reordering of the radiology work studies in the radiologist work list such that a radiology work study with a first service level priority is ordered above a radiology work study with a lower service level priority.

6. The method of claim 5, wherein the updating of the service level priority is based on an amount of time remaining with regard to a service level commitment associated with the radiology work study.

7. The method of claim 6, wherein the service level commitment comprises a contractual guarantee for a radiologist to complete the radiology work study within a predefined amount of time.

8. A system, comprising at least one processor and memory, for assigning work studies to be read within a work list, the method comprising:
a module to receive a radiologist work list comprising a plurality of radiology work studies;
a module to receive at least one parameter associated with the radiology work studies in the radiologist work list, the at least one parameter comprising a service level priority for the radiology work studies; and
a module to assign an order for the radiology work studies in the radiology work list based on the at least one parameter associated with the radiology work studies.

9. The system of claim 8, wherein the order of the radiology work studies in the radiologist work list is assigned such that the order specifies a priority for the radiology work studies to be read by a radiologist associated with the radiologist work list.

10. The system of claim 8, wherein the ordering of the radiology work studies is further based on a parameter of the radiologist comprising at least one of a subspecialty of the radiologist, a professional credential of the radiologist, and a location of the radiologist.

11. The system of claim 8, wherein the service level priority associated with each of the radiology work studies is updated based on an elapsed time associated with each of the radiology work studies.

12. The system of claim 11, wherein an updating of the service level priority associated with each of the radiology work studies comprises a reordering of the radiology work studies in the radiologist work list such that a radiology work study with a first service level priority is ordered above a radiology work study with a lower service level priority.

13. The system of claim 12, wherein the updating of the service level priority is based on an amount of time remaining with regard to a service level commitment associated with the radiology work study.

14. The system of claim 13, wherein the service level commitment comprises a contractual guarantee for a radiologist to complete the radiology work study within a predefined amount of time.

15. A non-transitory computer readable medium carrying one or more instructions for assigning work studies to be read within a work list, wherein the one or more instructions, when executed by one or more processors, causes the one or more processors to perform the steps of:
receiving a radiologist work list comprising a plurality of radiology work studies;
receiving at least one parameter associated with the radiology work studies in the radiologist work list, the at least one parameter comprising a service level priority for the radiology work studies; and
assigning, by a computer, an order for the radiology work studies in the radiology work list based on the at least one parameter associated with the radiology work studies.

16. The non-transitory computer readable medium of claim 15, wherein the order of the radiology work studies in the radiologist work list is assigned such that the order specifies a priority for the radiology work studies to be read by a radiologist associated with the radiologist work list.

17. The non-transitory computer readable medium of claim 15, wherein the ordering of the radiology work studies is further based on a parameter of the radiologist comprising at least one of a subspecialty of the radiologist, a professional credential of the radiologist, and a location of the radiologist.

18. The non-transitory computer readable medium of claim 15, wherein the service level priority associated with each of the radiology work studies is updated based on an elapsed time associated with each of the radiology work studies.

19. The non-transitory computer readable medium of claim 18, wherein an updating of the service level priority associated with each of the radiology work studies comprises a reordering of the radiology work studies in the radiologist work list such that a radiology work study with a first service level priority is ordered above a radiology work study with a lower service level priority.

20. The non-transitory computer readable medium of claim 19, wherein the updating of the service level priority is based on an amount of time remaining with regard to a service level commitment associated with the radiology work study.
